(19) United States
(12) Patent Application Publication
(10) Pub. No.: US 2025/0090024 A1
(43) Pub. Date: Mar. 20, 2025

(54) CONDITIONAL VARIATIONAL AUTOENCODER FOR FUNCTIONAL CONNECTIVITY ANALYSIS OF ASD FMRI DATA

(71) Applicant: The George Washington University, Washington, DC (US)

(72) Inventors: Mariia Sidulova, Washington, DC (US); Chung Hyuk Park, Washington, DC (US)

(21) Appl. No.: 18/890,038

(22) Filed: Sep. 19, 2024

Related U.S. Application Data
(60) Provisional application No. 63/539,176, filed on Sep. 19, 2023.

(51) Int. Cl. A61B 5/00; A61B 5/055; A61B 5/16; G01R 33/48

(52) U.S. Cl. CPC A61B 5/0042; A61B 5/055; A61B 5/16; A61B 5/4842; A61B 5/7246; A61B 5/7267; A61B 5/7485; G01R 33/4806; A61B 2576/026

(57) ABSTRACT

Generative models, such as Variational Autoencoders (VAEs), are increasingly employed for atypical pattern detection in brain imaging. During training, these models learn to capture the underlying patterns within “normal” brain images and generate new samples from those patterns. Neurodivergent states can be observed by measuring the dissimilarity between the generated/reconstructed images and the input images. The present system and method leverages VAEs to conduct Functional Connectivity (FC) analysis from functional Magnetic Resonance Imaging (fMRI) scans of individuals with Autism Spectrum Disorder (ASD), aiming to uncover atypical interconnectivity between brain regions. Multiple VAE architectures (Conditional VAE, Recurrent VAE, and a hybrid of CNN parallel with RNN VAE) establish the effectiveness of VAEs in application FC analysis. Given the nature of the disorder, ASD exhibits a higher prevalence among males than females. Therefore, we introduced phenotypic data to improve the performance of VAEs and, consequently, FC analysis. The present CNN-based VAE architecture is more effective for this application than the other models.

### RELATED APPLICATION

This application claims the benefit of priority of U.S. Provisional Application No. 63/539,176, filed Sep. 19, 2023, the entire content of which is relied upon and incorporated herein by reference in its entirety.

### GOVERNMENT LICENSE RIGHTS

This invention was made with Government support under Grant/Contract No. 170774 awarded by NSF. The Government has certain rights in this invention.

### BACKGROUND

Autism Spectrum Disorder (ASD) is a neurodevelopmental disorder/condition in which individuals experience difficulties in social communication and interaction and exhibit limited or repetitive behaviors and interests. Additionally, autistic individuals may have alternative learning styles, movements, and attention patterns. Several studies have consistently shown that ASD is more commonly found in males than females, with an approximate ratio of 3 to 1. One of the approaches used to investigate neurodivergence associated with ASD is Functional Connectivity (FC) analysis of functional Magnetic Resonance Imaging (fMRI) data. FC analysis helps to examine statistical dependence between the activity of different brain regions based on their blood oxygenation levels measured by fMRI. Hence, FC represents the extent to which various brain regions exhibit synchronized activity over a period of time, which is commonly believed to be representative of the structural and functional organization of the brain.

Functional Connectivity (FC) studies in ASD have led to the development of two main theories about the connectivity of the brains of individuals with ASD: under-connectivity and over-connectivity. Under-connectivity is defined as a decrease in brain activity between brain regions compared to a neurotypical population. Conversely, over-connectivity is understood as higher statistical correlations between different areas of the brain appearing in affected individuals compared to unaffected individuals. Finally, as more recent studies indicate, it is more likely that both over- and under-connectivity patterns are present in the brains of individuals with ASD.

Traditional methods for FC analysis include Seed-Based Correlation Analysis (SCA) [1], Independent Component Analysis (ICA) [2], graph theory-based analysis [3], clustering-based approaches [4], dynamic connectivity analysis [5], Granger causality analysis [6], and dynamic causal modeling [7]. While these approaches have helped uncover neurodivergent patterns in fMRI data, they entail certain limitations, such as inherent biases or limited interpretability. Several inconsistencies have been reported in studies using these methods when examining functional connectivity patterns in fMRI in ASD. The discrepancies are mainly attributed to the varied age and sex compositions within the study samples and the diverse nature of ASD. Notably, an apparent trend of under-representation of females with ASD in FC studies of fMRI can be seen. Limited interpretability arises from technical constraints inherent to each of these methods.

This disclosure is structured as follows: we discuss the pertinent literature on traditional FC methods and the utilization of Variational AutoEncoder (VAEs) (FIG. 1A) and Conditional Variational AutoEncoder (CVAEs) (FIG. 1B) in the fMRI domain. Additionally, we provide a concise overview of previously investigated FC divergences in ASD. Subsequently, we introduce the dataset, explain the data preprocessing techniques employed, elaborate on the VAE and CVAE architectures utilized, and detail our FC analysis approach. We then present our findings, and draw comparisons between our findings and those of previous studies.

### 1. BACKGROUND WORKS

### 1.1. Traditional Approaches to FC Analysis

Various methods have been developed to examine brain functional connectivity using fMRI data [16], which includes Seed-Based Correlation Analysis (SCA) [1], independent component analysis (ICA) [2], and graph-theory based analysis [3]. SCA involves selecting a Region of Interest (ROI) and computing its correlations with other brain regions over time series. High correlations indicate over-connectivity, and low correlation under-connectivity. However, SCA can potentially introduce bias due to ROI selection, overlooking important connectivity patterns outside the chosen regions [11]. On the other hand, ICA is a data-driven, multivariate method that decomposes fMRI data into spatially independent components, each representing a unique spatial pattern associated with a distinct time course [2,12]. ICA has been applicable in revealing lower-level spatial and temporal patterns in brain connectivity. Nevertheless, the drawback of ICA analysis is that the signal from a single brain region may appear in multiple components within lower-dimensional space, complicating the identification of high-level correlations.

Graph theory provides a framework for investigating local and global connectivity patterns. However, effectively capturing the temporal dynamics inherent in fMRI data presents a significant challenge. More advanced traditional approaches to Functional Connectivity Analysis (FC) include clustering-based approaches [4], dynamic connectivity analysis [5], Granger causality analysis [6], and dynamic causal modeling [7]. Most studies using traditional methods have focused on male fMRI data with ASD, and there has been a lack of research specifically exploring females with ASD. When the dataset is imbalanced, SCA, ICA, and graph-based analyses face several challenges. For example, SCA is often used to compare connectivity patterns between different subgroups; thus, an imbalance in the studied data can influence the statistical power and robustness of the comparisons. In ICA, while the analysis is not inherently affected by class imbalance, subsequent classifiers that use ICA-derived features may favor the majority class, affecting classification performance. In graph-based methods, graph construction could also be hindered by the greater presence of certain populations. Therefore, there is a need for an approach that encompasses both the spatial and temporal distribution of the data and is robust to under-representations in the dataset.

### 1.2. Application of VAEs in fMRI Domain

FIG. 1A shows an example VAE (Variational AutoEncoder). There has been a surge in the utilization of VAEs to identify brain connectivity patterns within affected populations or fMRI signal patterns related to specific tasks. VAEs offer the advantage of allowing for the studying of both low- and high-level features of fMRI data, setting them apart from techniques such as ICA and SCA. Several papers used VAEs to extract meaningful features to classify the data [13-15]; some studies also researched the abilities of VAEs to identify task-related activities [16,17], and finally, some utilized VAEs for FC analysis of the fMRI data [18,19].

The most closely related is the paper by Zuo et al., in which the researchers utilized a disentangled VAE to identify structural and functional connectivity differences between control, individuals with early mild cognitive impairment (MCI), and individuals with late mild cognitive impairment [18]. Using a graph convolutional VAE, researchers have identified under- and over-connectivity patterns associated with the progression of MCI. Likewise, another study by Choi et al. applied a Deep Neural Network (DNN)-based VAE to analyze connectivity patterns in ASD [19]. The study has also presented under- and over-connectivity patterns correlated with the full-scale IQ scores.

A considerable number of encoder and decoder architectures have been studied in the application of fMRIs, which vary depending on the main objective of the application. However, the most common architectures include convolutional layers (CNN), recurrent layers (RNN), and a combination of the two in sequence and parallel. CNN layers have proven to be helpful in identifying spatial correlations; however, the temporal patterns of the decoded data are not meaningful since the convolution is not capable of capturing the temporal dynamics. And vice versa, recurrent layers have shown to have better temporal feature extraction, but spatial patterns could not be well preserved. Therefore, we believe that there is a need to evaluate different model architectures.

### 1.3. Application of CVAEs in fMRI Domain

FIG. 1B shows an example of a Conditional Variational AutoEncoder (CVAE). CVAEs are an extension of the VAEs and incorporate additional information into the generative model [9]. The generative process in a CVAE is improved by considering additional information, such as class labels, attributes, or any other relevant data. Conditional variables are then passed into both the encoder and decoder parts of the VAE. Therefore, the encoder takes the input data and associated conditional variables and maps them to a distribution in the latent space. The decoder then uses the sampled latent distribution from the encoder along with the conditional variables to reconstruct the input data point. By adding additional information to the generation process, CVAEs allow for more targeted and controlled data generation. In the context of fMRI imaging, CVAEs have been used for image synthesis and data augmentation [20], brain image segmentation [21], classification [22], and connectivity network detection [23]. The most closely related to our study is the study by Wang et al., which used adverse CVAE to identify high-level neurodivergent patterns associated with Alzheimer's Disease (AD) in fMRI data [24]. Researchers have demonstrated that applying conditions to the network helps reduce the effect of age and sex bias in the latent vectors. Another paper that used CVAE is the study by Gao et al., where researchers integrate age and sex attributes through an attention mechanism that optimizes VAE for the classification of brain connectivity from fMRI data of individuals with attention-deficit/hyperactivity disorder from multiple sites [25]. The study showed that phenotypic information has improved learning discriminative embedding and helped identify affected brain regions functionally by reconstructing the latent features.

### 1.4. Functional Connectivity in ASD

The most commonly studied brain networks in ASD include Default Mode Network (DMN), limbic, visual, somatomotor, and salience networks. The regional components of each of these networks have a tendency to slightly change study by study. The DMN is a large-scale brain network that is most active during rest periods or when the mind is wandering [26]. It is involved in various cognitive processes such as self-thinking, episodic memory recovery, and social cognition [26]. In most studies, the DMN includes regions such as the medial prefrontal cortex, the posterior cingulate cortex, and the medial temporal lobes. The limbic network is a group of interconnected structures that play a critical role in emotion, motivation, and memory processing [27]. The limbic network is closely associated with the management of emotional responses, the processing of reward and punishment, and the formation and recovery of memories. Key structures in the limbic system include the amygdala, hippocampus, and cingulate gyrus [28]. The visual network is responsible for processing visual stimuli, and its nodes are located primarily in the occipital lobe [29]. The somatomotor network is involved in the planning, enactment, and management of voluntary movements. It includes the primary motor cortex, the supplementary motor area, and the primary somatosensory cortex, all located in the frontal and parietal lobes. Finally, the salience network is a large-scale brain network that is involved in catching and focusing attention to relevant internal and external stimuli [30]. Key regions within the salience network include the anterior insula and the dorsal anterior cingulate cortex [31].

Previous findings suggest that under-connectivity between various brain networks is associated with social impairments and deficits observed in ASD. Most under-connectivity patterns were associated with DMN, including decreased interconnectivity between DMN-limbic, DMN-visual, and DMN-somatomotor. For example, in the study by Abrams et al., the researchers reported under-connectivity between DMN (pSTS with orbitofrontal, temporal lobe) and limbic networks (amygdala), suggesting that ASD individuals experience a less pleasant response to human voice processing [32]. Under-connectivity between the DMN (Precuneus (PrC)) and the visual cortex has also been previously reported [33]. However, the study reported that this under-connectivity pattern was not found to be related to socio-behavior deficits. Finally, under-connectivity between DMN and several regions in somatomotor has also been reported in multiple studies [34,35].

Over-connectivity patterns are primarily associated with salience networks. For example, a study by Green et al. has demonstrated the over-connectivity of the salience network with sensory processing areas, such as the visual and limbic networks, in individuals with ASD. It is believed that this over-connectivity may contribute to heightened responsiveness to irrelevant stimuli and deficits in social interactions [36]. DMN-salience network was shown to have higher interconnectivity in ASD subjects compared to Typically Developing (TD) in work by Yerys et al. [34], which has been hypothesized to be attributed to the ability to switch between intra-person and extra-person processing.

A handful of studies specifically looked into the difference between female and male functional connectivity. One of the few studies of specifically sex-related differences revealed that commonly associated DMN hypoconnectivities are primarily present in male populations [37]. Increased connectivity in the female population compared to males has also been supported by the studies by Lawerence et al. and Smith et al. [39].

Examples/papers on diagnosis based on FC analysis:
J. Wang. Q. Wang, H. Zhang, J. Chen, S. Wang and D. Shen, “Sparse Multiview Task-Centralized Ensemble Learning for ASD Diagnosis Based on Age- and Sex-Related Functional Connectivity Patterns,” in IEEE Transactions on Cybernetics, vol. 49, no. 8, pp. 3141-3154 August 2019, doi: 10.1109/TCYB.2018.2839693.
Wee, Chong-Yaw, Pew-Thian Yap, and Dinggang Shen. “Diagnosis of autism spectrum disorders using temporally distinct resting-state functional connectivity networks.” CNS neuroscience & therapeutics 22.3 (2016): 212-219.
Holiga, Štefan, et al. “Patients with autism spectrum disorders display reproducible functional connectivity alterations.” Science Translational Medicine 11.481 (2019): eaat9223. Karunakaran, P., and Yasir Babiker Hamdan. “Early prediction of autism spectrum disorder by computational approaches to (MRI analysis with early learning technique.” Journal of Artificial Intelligence 2.04 (2020): 207-216.

Examples/papers on progress monitoring based on FC analysis or fMRI data include Binnewijzend, Maja A A, et al. “Resting-state fMRI changes in Alzheimer's disease and mild cognitive impairment.” Neurobiology of aging 33.9 (2012): 2018-2028. And. Yang, Fu-Chi, et al. “Altered hypothalamic functional connectivity in cluster headache: a longitudinal resting-state functional MRI study.” Journal of Neurology. Neurosurgery & Psychiatry 86.4 (2015): 437-445.

### SUMMARY

To address the issues of limited interpretability and under-representation, a system and method is provided with a novel approach to FC analysis of fMRI data using Variational AutoEncoders and Conditional Variational AutoEncoders. The Variational AutoEncoder (VAE) is a deep generative model that learns to encode data into a low-dimensional latent space and then decodes low-dimensional features back to the original data [8]. The Conditional Variational Autoencoder (CVAE) is an extension of the standard VAE, which incorporates conditional information, such as additional class features or attributes, into the generative model to enable targeted data synthesis [9]. This disclosure applies three different VAE architectures for FC analysis for individuals with ASD. We then apply phenotypic data to VAEs to reduce sex-related bias. For a more quantitative and structured analysis, we have employed three commonly used VAE architectures in the fMRI domain: Convolutional Neural Network (CNN), Recurrent Neural Network (RNN), and a hybrid model combining CNN and RNN in parallel. Our evaluation of the VAE and CVAE includes comparing the performance in the reconstruction of neurotypical samples and the efficacy in conducting FC analysis for fMRI samples of individuals with ASD. Our evaluation compares the identified FC divergences between female and male populations for both VAE and CVAE. We aim to provide a structural and systemic investigation with diverse AE architecture variations in the fMRI domain, specifically addressing the issues of dynamic processing of highly complex brain imaging data and sex under-representation with statistical modeling.

Current practice is the doctor's visual assessment or traditional machine learning algorithms with statistical analysis models. This IP uses generative AI to model resting state fMRI signals and then detects changes using the generative models to analyze connectivity changes, taking into account other features such as age or gender.

The present disclosure Computational model to process fMRI data for neural connectivity analysis and ASD diagnosis/progress estimations.

These and other objects of the disclosure, as well as many of the intended advantages thereof, will become more readily apparent when reference is made to the following description, taken in conjunction with the accompanying drawings. This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

FIGS. 1A, 1B illustrate the difference between VAE (FIG. 1A) and CVAE (FIG. 1B). In CVAE, both the encoder and decoder part receive conditional attributes; in the present system, it is an embedding of age, sex, and group label.

FIGS. 2A-2C show details on different structures of the model architecture for FC analysis with fMRI data. FIG. 2A is the overall signal processing framework. FIG. 2B is a CNN CVAE. FIG. 2C is a RNN CVAE. FIG. 11A is a Hybrid CVAE with CNN and RNN in parallel.

FIG. 3 is a summary of phenotypic data is presented. In particular, the number of male samples is higher than that of females in both subjects with typically developing ASD and subjects with ASD.

FIGS. 4A-4C are a summary of functional connectivity analysis steps. FIG. 4A shows process neurodivergent samples from the validation subset through VAE or CVAE. Adjust the condition to the target in CVAE. FIG. 4B compute pairwise connectivity between networks. FIG. 4C perform a two-sided Welch t-test and visualize statistically significant results using a chord diagram.

FIG. 5 are sample reconstruction of parcels versus time matrix for a neurotypical control sample from validation subset. LH: Left Hemisphere, RH: Right Hemisphere, Vis: Visual, SM: Somatomotor, Lim: Limbic, Sal: Salience, Def: Default.

FIG. 6 is sample reconstructions of one parcel for the neurotypical control sample from validation subset. PCC and MSE are also stated for the displayed parcel reconstruction.

FIG. 7 shows summary of mean distributions of the latent space for validation subsets for each model. T-test significance is also reported on each of the subplots.

FIG. 8 shows statistically significant results of FC analysis presented as the chord diagram from VAE systems (two-sided Welch's, p<0.05). The bluish color of the lines indicates lower connectivity, while yellowish colors represent higher connectivity of ASD samples compared to neurotypical-like synthetic samples. The top row displays combined results for both female and male populations, the middle row focuses on the male population only, and the bottom row pertains to female samples.

FIG. 9 shows statistically significant results of FC analysis presented as the chord diagram from VAE systems (two-sided Welch's, p<0.05). The bluish color of the lines indicates lower connectivity, while yellowish colors represent higher connectivity of ASD samples compared to neurotypical-like synthetic samples. The top row displays combined results for both female and male populations, the middle row focuses on the male population only, and the bottom row pertains to female samples.

FIGS. 10A-10C are hardware architecture diagrams. In FIG. 10B, the color of the box corresponds to color of the functional flow diagram in FIG. 12.

FIGS. 11A-11C show more illustrative examples of the (FIG. 11A) model architecture (FIG. 11B). Output analysis (FIG. 11C) Final visualization of the results.

FIG. 12 is an operational flow diagram.

### DETAILED DESCRIPTION

In describing the present disclosure illustrated in the drawings, specific terminology is resorted to for the sake of clarity. However, the present disclosure is not intended to be limited to the specific terms so selected, and it is to be understood that each specific term includes all technical equivalents that operate in a similar manner to accomplish a similar purpose.

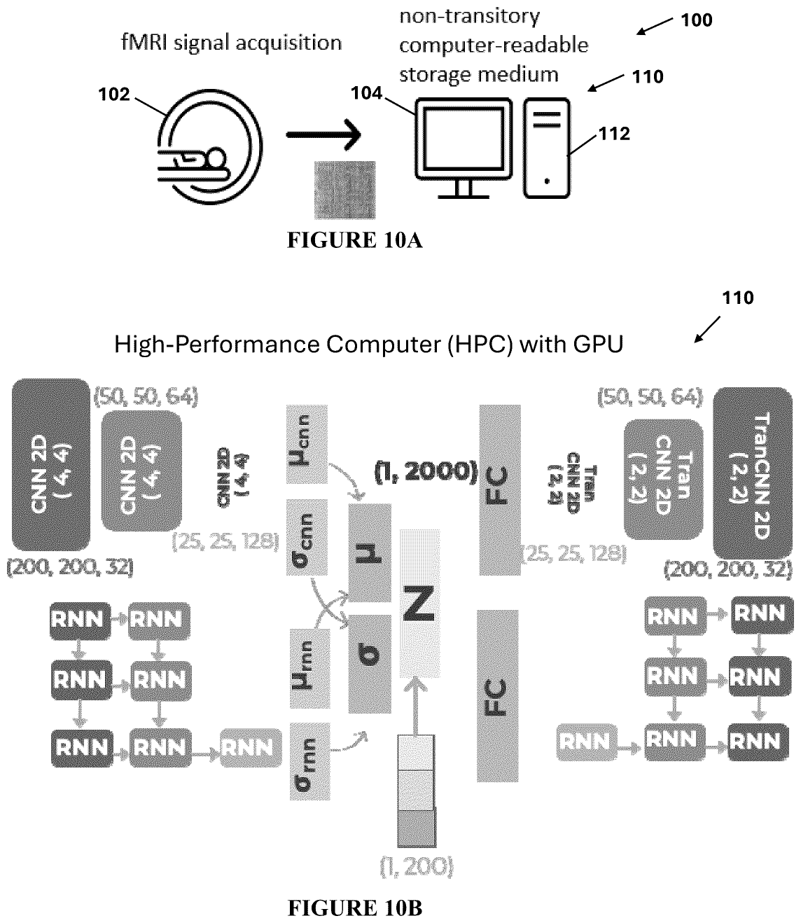

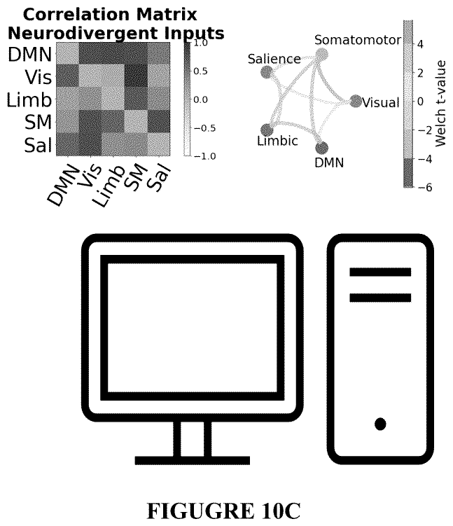

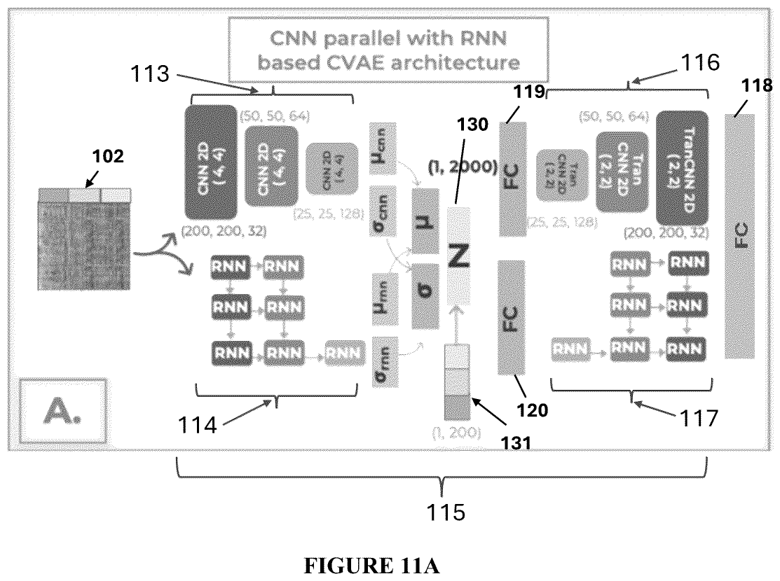

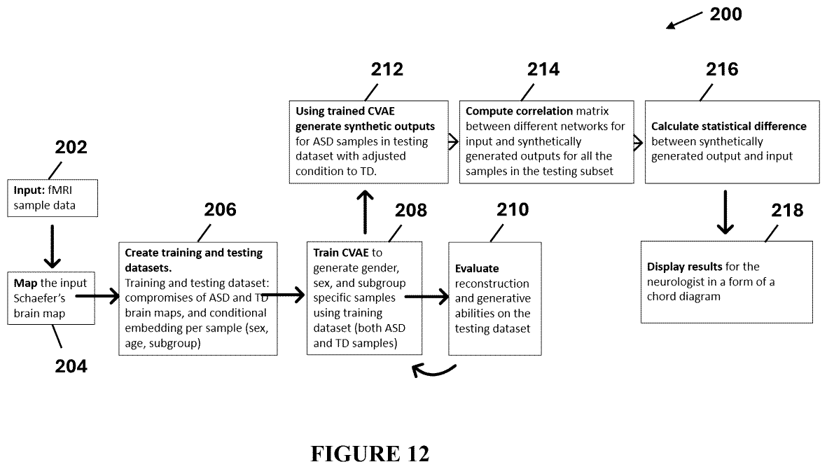

Turning to the drawings, FIGS. 10A-10C show a non-limiting example illustrative embodiment of an image classification and inventory management system 100, especially for use in identifying neurodivergencies and particularly ASD neurodivergencies. The system 100 includes an image source 102, a processing device 110 (for example, here shown as high-performance computer (HPC)) (FIG. 10B), image storage device 112, and an output device 104 (for example, here shown as a display device or monitor). The image source 102 can be any device that captures an image of a patient, such as for example a camera, x-ray machine, electrocardiogram (ECG) device, MRI, computerized tomography (CT) device. The image source 102 provides the image data to the image storage device 112, which stores the image data. The image storage device 112 can be located at the processor 110 (as shown), or at the image source 102, or remote from the processor 110 and/or the image source 102. The HPC 110 is configured to retrieve the image data from the storage device 112, and performs certain operations, for example as shown in FIG. 12. The HPC is further configured to generate a display output, which is displayed on the display device.

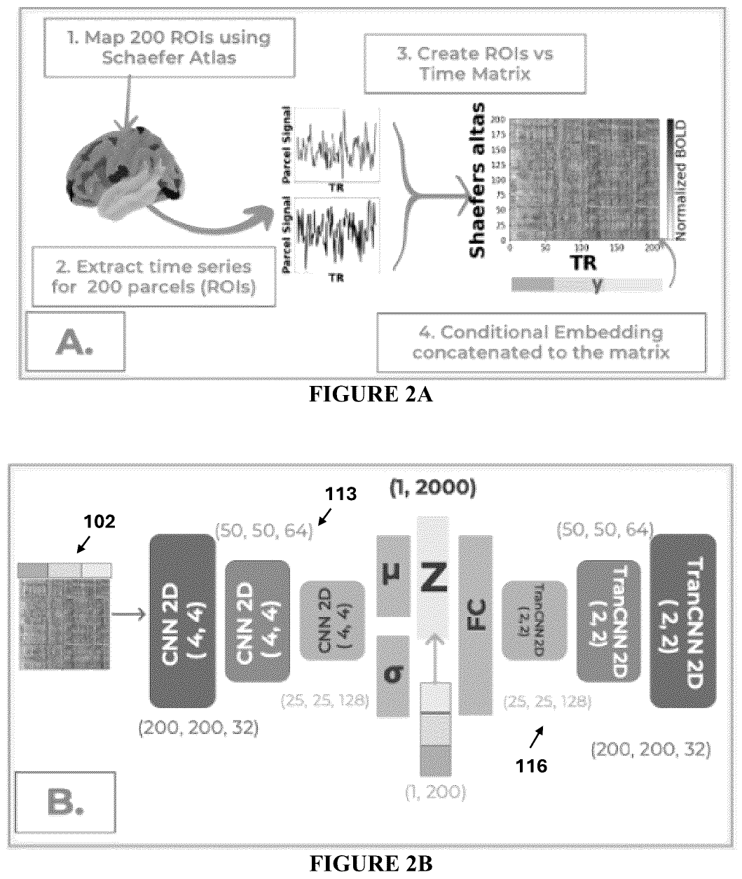

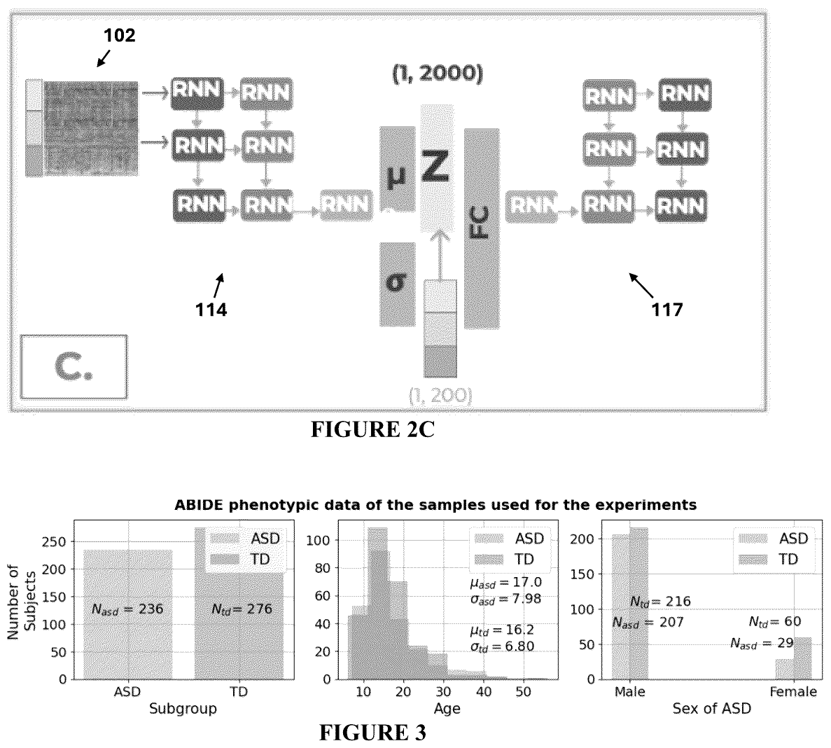

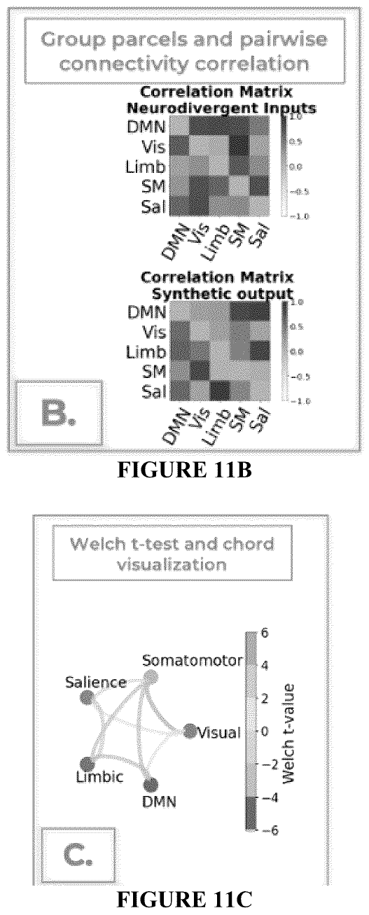

FIG. 11A shows one example of the processing device 110, and further details are shown and described, for example, with respect to in FIGS. 2A-2C. The processor 110 comprises a CVAE architecture 115 that includes an encoder and a decoder. The encoder includes CNN encoder layers 113, and RNN encoder layers 114. The decoder includes CNN decoder layers 116, and RNN decoder layers 117. The CNN encoder layers 113 are arranged in parallel with the RNN encoder layers 114. For all CNN 113 branches of CVAEs, the CNN encoder has three convolution layers arranged in sequence, with a first CNN layer having 32 filters, a second CNN layer having 64 filters, and a third CNN layer having 128 filters, respectively, followed by a fully-connected layer. The CNN layers 113 in the encoder, process spatial features of the fMRI image data and reduce dimensions over each layer to produce lower-dimension feature sets and provide as inputs to the latent Z space 130.

Thus, with reference to FIGS. 2B, 11A, the first CNN encoder layer is connected to the fMRI image source 102 (e.g., storage 112), receives the fMRI input image data from the fMRI image source and generates a first CNN encoder fMRI image data output. The second CNN encoder layer is connected to the first CNN encoder layer, receives the first CNN encoder fMRI image data output from the first CNN encoder layer, and generates a second CNN encoder fMRI image data output. The third CNN encoder layer is connected to the second CNN encoder layer, receives the second CNN encoder fMRI image data output from the second CNN encoder layer, and generates a third CNN encoder fMRI image data output.

As shown in FIGS. 2C, 11A, the RNN encoder 114 is connected to the fMRI image source 102 (e.g., storage 112), receives the fMRI input image data from the fMRI image source, and generates an RNN encoder fMRI image data output. The RNN encoder modules 114 in the encoder 114 process the temporal features of the fMRI data and map key temporal features in reduced space to provide inputs to the latent Z space 130 as well. The CVAE decoder is trained to reconstruct the Z space latent vector of size (1,2000) and the conditional variable with the size of (1,200) to reconstruct the synthetic fMRI data through the parallel architecture of the CNN decoder 116 and RNN decoder 117 in the decoder part of the CVAE.

The CNN encoder 113 and RNN encoder 114 are connected to the Fully Connected input of a Fully Connected layer 119, 120, respectively, of the CVAE Decoder block. The Fully Connected layer 119 input receives the third CNN encoder fMRI image data output and the third RNN encoder fMRI image data output.

The CNN decoder 116 and RNN decoder 117 are connected to the output of the Fully Connected layers 119, 120, respectively, of the CVAE Decoder block (that includes the CNN decoder 116 and the RNN decoder 117 to generate the synthetic fMRI data output.

The CNN decoder 116 is comprised of the Fully Connected layer 119 followed by transposed convolution layers with 128, 64, and 32 filters (three layers in the embodiment of FIG. 11A). Batch normalization and the leaky ReLU activation functions are utilized to generate normalized input distribution to the network layers with reduced loss of information (this happens inside the network so not visualized). Thus, the first CNN decoder layer is connected to the latent Z space 130, receives the latent space vector encoded from the CVAE encoder layer 113 (the third CNN output) that has gone through the dimension reduction process from the encoder part from the input fMRI data and the conditional embeddings 131 (e.g. age, sex, and neurodivergent label), and generates a first CNN decoder synthetic output that extrapolates from the lower-dimension latent space vector in 130 toward higher dimensional synthetic data. The second CNN decoder layer is connected to the first CNN decoder layer, receives the first CNN decoder synthetic data output from the first CNN decoder layer, and generates a second CNN decoder synthetic data output. The third CNN decoder layer is connected to the second CNN decoder layer, receives the second CNN decoder synthetic data output from the second CNN decoder layer, and generates a third CNN decoder synthetic data output.

The RNN decoder 117 is connected in parallel with the CNN decoder 116. The RNN branch encoder 114 contains three unidirectional Long-Short Term Memory (LSTM) layers followed by a Fully Connected layer 118. The RNN decoder 117, includes a Fully Connected layer 120 followed by three LSTM layers. Latent features are fused using element-by-element multiplication. The RNN decoder 117 is connected to the latent Z space 130 output, receives latent space vector encoded from the CVAE encoder layer (the third decoder output) that has gone through the dimension reduction process from the encoder part from the input fMRI data and the conditional embeddings 131 (e.g. age, sex, and neurodivergent label), and generates a RNN decoder synthetic data output. The total number of latent features in the latent Z space 130 is equal to 2000.

The outputs from the CNN decoder 116 and the RNN decoder 117 are connected to a Fully Connected layer 118 of the CVAE decoder output layer. The Fully Connected layer 118 produces synthetic fMRI data. For example, the CVAE 115 (conditioned on sex, age, and neurodivergence subgroup label) can be applied to fMRI data that has been mapped to a predefined brain atlas. The CVAE 115 can be used to generate neurodivergent synthetic fMRI data corresponding to the conditions of sex, age and neurodivergence subgroup label using the CVAE model. In addition, the CVAE 115 can be used to generate at the processing device, non-neurodivergent synthetic fMRI data corresponding to sex and age conditions using the CVAE model. The output from the CVAE 115 can then be used for functional connectivity analysis to identify differences between the neurodivergent synthetic fMRI data and the non-neurodivergent synthetic fMRI data to identify neurodivergences in the patient fMRI data. Of course, other suitable configurations for the CVAE 115 can be utilized, within the spirit and scope of the present disclosure. In some embodiments, a VAE can be utilized.

FIG. 11B shows the display data output by the processing device 110, for example having group parcels and pairwise connectivity correlation. The displayed chord diagrams can be used by physician to evaluated the specific neurodivergent connections for individuals. The chord diagram displays the connections that are either statistically significantly lower than in typically developing population, or vice versa that are significantly higher than in neurotypical. Clinical significance of the identified neurodivergences and relation to the social deficits and other symptoms of ASD can be evaluated by physician, by observing the over-connectivity (OC) and under-connectivity (UC) in functional connectivity between the brain regions along with clinical observations.

FIG. 11C shows a visualization of the results that are displayed on the display device. The chord graph in FIG. 11C visualizes the OC and UC identified in the correlation matrix from FIG. 11B into colored scale for more intuitive visualization.

FIG. 12 is a functional flow diagram 200 of the system 100. The input steps 202, 204 206 are performed by FIG. 10A; the atlas mapping steps (training and evaluation of CVAE) 208, 210 are performed by the processor 110 (FIG. 10B); and the further analysis using trained CVAE (creating training and testing datasets) steps 212, 214, 216, 218 are performed by the processor 110 (FIG. 10C).

Accordingly, starting at step 202, the fMRI sample data is input by the image source 102 and optionally stored in the image storage 112. At step 204, the processor 110 maps the input fMRI image data, for example based on Schaefer's brain map. An atlas is what is used to get a brain map, parses the fMRI brain image data into specific regions, and is used here reduce the data dimensionality. The raw fMRI signals (sample signals shown in the middle of FIG. 2A) is input to the processor 110 to map into Schafer's atlas volumetric parcellation to produce data as visualized in the input stage of FIG. 11A (as well as output stage in FIG. 2A).

At step 206, the processor 110 creates training and testing datasets, which compromises of neurodiverse brain signals (e.g., brain signals from ASD and TD subjects), and conditional embedding per sample (sex, age, subgroup). The training and testing data sets are randomly selected from the total dataset to provide balanced training and proper evaluation. The conditional embeddings 131 are specific to each subject, so they constitute part of the conditional variable inputs to the CVAE.

At step 208, the processor 110 trains the CVAE to generate gender, sex and subgroup specific samples using training dataset (both ASD and TD samples).

At step 210, the processor 110 evaluates reconstruction and generative abilities on the testing dataset to validate the reconstruction rate of synthetic brain signals. The reconstruction rate is calculated with generative loss in comparison to the input brain signals.

At step 212, the processor 110, at the decoder 116, 117 (FIG. 11A) uses the trained CVAE data to generate synthetic outputs for ASD samples in testing dataset with adjusted condition to TD. The synthetic outputs models generalized synthetic brain signals, so the differences between synthetic outputs from ASD condition input and TD condition input will generate the functional connectivity differences. The condition generation of samples is performed with the components that are depicted with 3 rectangles.

At step 214, the processor 110, from the outputs of the decoder 116, 117, computes correlation matrix between different networks for input and synthetically generated outputs for all the samples in the testing subset. The correlation matrix explains the OC and UC as visualized in FIG. 11B.

At step 216, the processor 110 calculates the statistical differences between the synthetically generated output and input. In the FIG. 11A, the output from decoder components 116, 117 is subtracted from the input 113, 114.

At step 218, the processor 110 displays the results of the neurologist in a form of a chord diagram on the display device 104, as shown in FIG. 11C, which visualizes the OC and UC to explain the functional connectivity differences in ASD brain signals.

On the clinical front, this system 100 and method 200 significantly enhances early diagnosis and intervention strategies by providing more accurate and data-driven insights into ASD. Clinicians can leverage the model's predictions to identify atypical neurological patterns, enabling them to tailor treatment plans more effectively and improve the overall quality of care for individuals on the autism spectrum. Moreover, the system can streamline the diagnostic process, reducing the time and cost associated with traditional assessment methods.

This ML system also opens new avenues for research, pharmaceutical development, and therapeutic interventions. Pharmaceutical companies can utilize the insights generated by the system to identify potential targets for drug development, ultimately leading to more targeted and effective treatments for ASD. Additionally, the system can aid in market segmentation and the development of specialized products and services to better meet the unique needs of individuals with ASD and their families, thus fostering innovation and growth in the commercial sector.

The disclosure includes many illustrative embodiments, some of which are mentioned here. In a first embodiment 1, a computer-implemented method is provided for identifying neurodivergences in individuals using functional fMRI data, comprising: mapping fMRI imaging data to predefined brain atlas; training a conditional variational autoencoder (CVAE) model using the extracted features, wherein the CVAE model conditions on sex, age, and subgroup label; generating desired healthy-like synthetic fMRI samples using the trained CVAE model; and performing functional connectivity analysis on the synthetic fMRI samples to identify neurodivergences in individuals with ASD. In a second embodiment, the functional connectivity analysis includes calculating correlation matrices based on the synthetic fMRI samples; and applying Welch t-test to the correlation matrices to identify significant differences in functional connectivity patterns between individuals with ASD and synthetically generated healthy-like samples.

In a third embodiment, the CVAE model is further configured to optimize latent space representations of the fMRI data to enhance the separability of individuals with ASD and healthy individuals. In a fourth embodiment, the CVAE model is trained using a deep learning architecture comprising multiple layers of neural networks. In a fifth embodiment, a computer system is provided for identifying neurodivergences in individuals, comprising: a data collection module for collecting fMRI data from individuals; a feature extraction module for extracting features from the fMRI data; a conditional variational autoencoder (CVAE) model trained to condition on sex, age, and diagnosis, configured to generate synthetic fMRI samples; a functional connectivity analysis module for performing functional connectivity analysis on the synthetic fMRI samples to identify neurodivergences in individuals. In a sixth embodiment, a non-transitory computer-readable storage medium has instructions that, when executed by a computer, cause the computer to perform the method of the first through fifth embodiments.

### 2. MATERIALS AND METHODS

### 2.1. Dataset—this Application Deals with fMRI Data, Stored in the Storage Device 112 and Processed with the Processing Module 110

The ABIDE-I (Autism Brain Imaging Data Exchange) dataset is a publicly available, large-scale collection of resting-state fMRI data of individuals with ASD [46]. The ABIDE-I dataset consists of 1035 rs-fMRI scans, including 505 individuals with ASD and 530 neurotypical control subjects. The data were collected from 17 different imaging sites, each with its own scanning protocol. The dataset has undergone various preprocessing steps, including motion correction, spatial normalization, and noise reduction, to ensure uniform data quality and comparability across different sites. However, different imaging sites had different default fMRI scanners; therefore, Repetition Time (TR), Echo Time (TE), and flip angle degree are varied across sites. The subset of scans with TR of 2000 (ms) from the ABIDE-I dataset has been extracted. Thus, for the present system, we have only used data samples collected from 9 out of 17 sites, resulting in 236 ASD samples, 276 typically developing samples. The subjects were then randomly split into training and testing sets. The training and testing sets consisted of 231 control and 235 neurodivergent samples and 35 and 41 samples, respectively. In FIG. 3, phenotypic data distributions for the studied data could be found. It could be noted that there are a higher number of male samples than females in both typically developing and neurodivergent subgroups.

### 2.2. Data Preprocessing

FIG. 12 shows the overall process where the Schaefer's 200-parcel functional deterministic atlas has been used for brain parcellation of the original fMRI scans, which divided the cerebral cortex into 200 distinct, non-overlapping regions based on the derived functional connectivity patterns (FIG. 2A) [41]. The resulting 200 parcels are distributed across both hemispheres and cover the entire cortex. Time-series data were extracted from each of the 200 parcels, resulting in a 2D matrix having signals from 200 parcels with 200 time steps (TR=2000 ms). As the length of scans varied across imaging sights, each scan was augmented into multiple samples using a sliding window of 200 time steps with a step size of 10 applied to each voxel per time matrix. The sliding window was then applied to each sample in training and testing subsets, resulting in disjoint 3472 neurotypical and 2973 neurodivergent samples for the training set and 364 and 364 samples for the testing set. The testing and training fMRI splitting, described in Section 2.1, have not been mixed during data augmentation to ensure fairness. Finally, the parcel versus time matrices were normalized to the range of 0 to 1.

### 2.3 Variational Autoencoder (VAE)

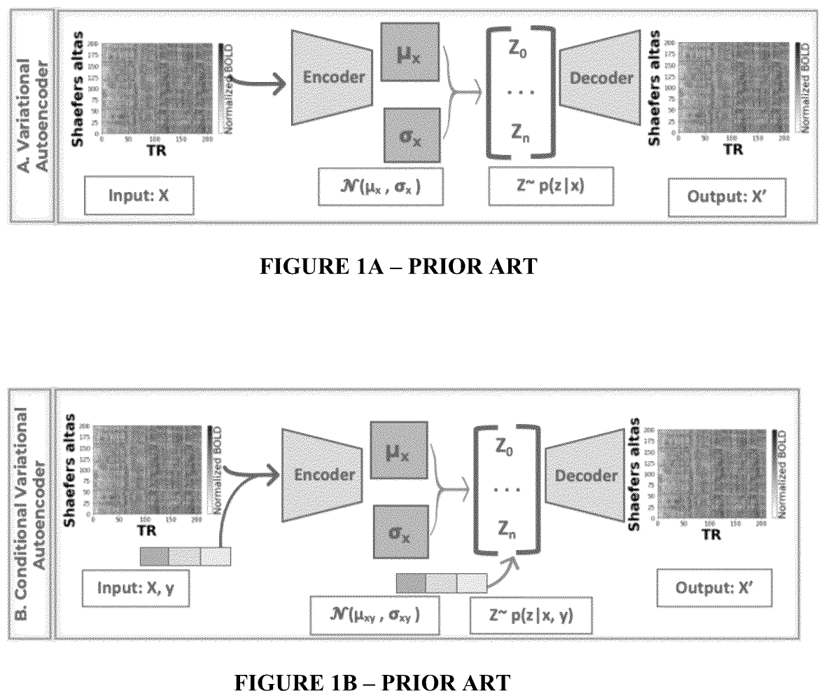

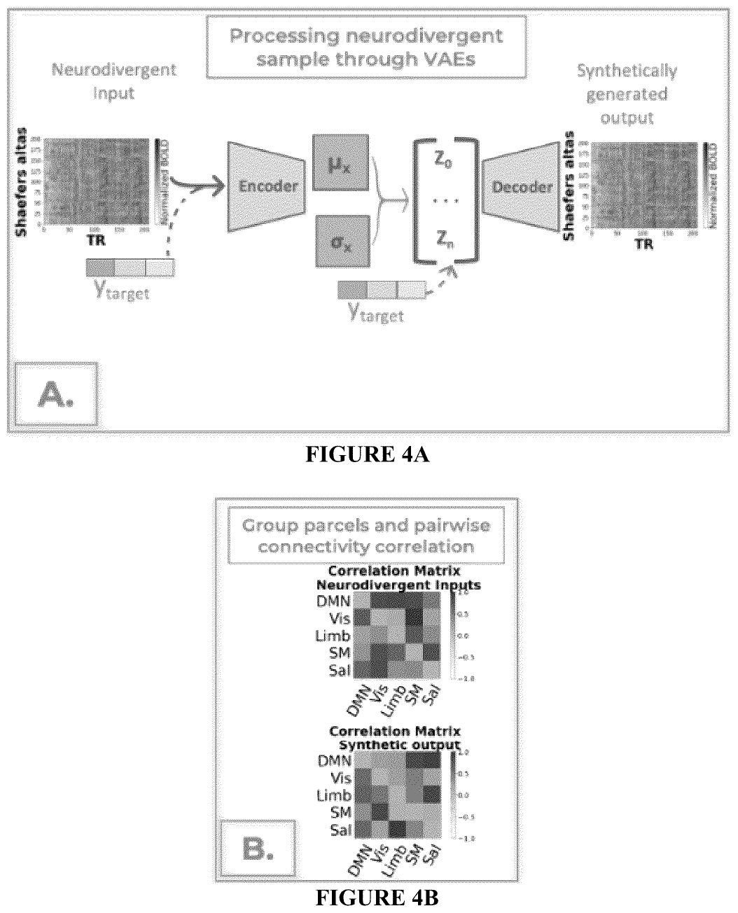

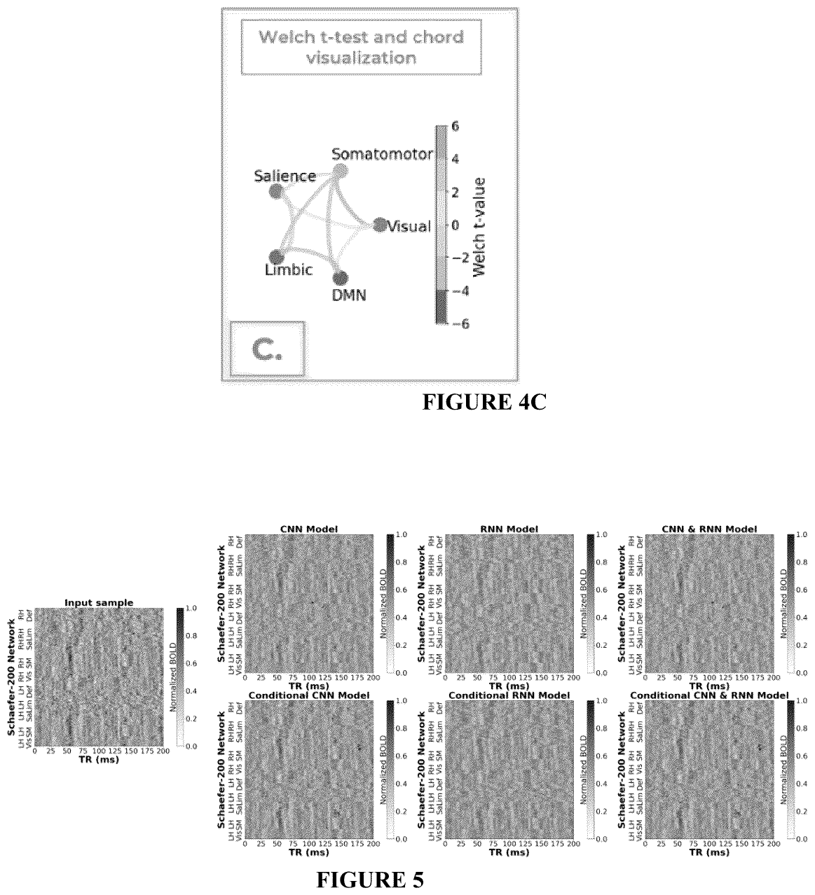

The Autoencoder (AE) (FIGS. 1A, 1B) is a type of neural network architecture commonly employed for capturing low-dimensional representations of fMRI data. The AE comprises an encoder and a decoder [13]. The encoder part of the AE transforms the input data into a set of low-dimensional latent variables, and the decoder part subsequently reconstructs those latent variables into the original data space [42]. During training, the encoder and decoder aim to minimize the reconstruction error between the input data and the reconstructed output [42]. A unique subtype of AEs is the Variational Autoencoder (VAE), as shown in FIG. 4A. Similar to the AE, the VAE also includes an encoder and a decoder, but the encoder maps the input data to a set of latent variables that are assumed to be drawn from a prior distribution. The decoder randomly samples from the latent distribution and learns to map these latent variables back to the original data space to reconstruct the sample. Sampling from a learned latent space and decoding these latent features into the original data space allows for the generation of new data samples.

In the present system, the VAE is deployed as a deep generative model using different architectures of the encoder g(x; φ) and the decoder f (z; θ). The encoder learns to compress the high-dimensional input (parcels versus time matrix) x into lower-dimensional latent representations z, and φ and θ are both hyperparameters of the networks. The VAE aims to learn a model for the true data distribution, denoted by p(z, x). The latent space dimensionality is denoted as d (i.e., z∈ [symbol] ^{d}). The variational posterior distribution is denoted by q(z, x), which is an approximation of the true posterior. The network is trained using the Evidence Lower Bound (ELBO) loss, having the reconstruction and KL divergence terms. The reconstruction term aims to ensure that the VAE can accurately reconstruct the input data, which is represented as the expected negative log-likelihood log p(x|z), where p(x|z) is modeled by the decoder part of the VAE. The KL divergence term is used to make the variational posterior distribution, q(z|x), as close to the prior distribution, p(z), as possible.

The ELBO loss, denoted as L_{ELBO}(x), can be written as:

$$\begin{matrix}{ℒ}_{ELBO} ( x ) = {E}_{q ( x , z )} [ log \frac{p ( z , x )}{q ( z \vert x )} ] \\ = {E}_{q ( z , x )} [ log p ( x \vert z ) + log p ( z ) - log q ( z \vert x ) ] \\ = {E}_{q ( z , x )} [ log p ( x \vert z ) ] - {D}_{KL} [ q ( z \vert x ) \vert p ( z ) ] ,\end{matrix} \qquad ( 1 )$$

During training, the encoder network g(x;φ) models the variational posterior distribution q(z|x). The encoder outputs the parameters of a Gaussian distribution, \tilde{μ} and log \tilde{σ}², which represent the mean and log-variance of the latent space distribution, respectively. Sampling from q(z|x) allows us to generate new data samples similar to those present in the training data distribution.

### 2.4 Conditional VAE

The system uses a CVAE for a more controlled fMRI sample reconstruction, as an example architecture shown in FIG. 11A. The CVAE is an extension of the VAE that allows the generation of data samples conditioned on certain attributes or labels [9]. In our CVAE design, both the encoder and decoder receive additional input variables, which is an embedding (denoted as y) containing age, sex (M or F), and subgroup (TD or ASD) labels, with the assumption that all conditions are statistically independent of each other. This can be viewed as concatenating the embedding to the input of the encoder x and the input of the decoder z. The changes made in comparison to the generative process of a VAE can be understood as introducing an identity function with respect to y into the model. In the CVAE, the encoder learns to extract hidden representations of an image x while taking into account conditional variables y (represented by the distribution q(z|x, y)). The decoder then translates this data representation in the form of (z, y) to the input space (i.e., p(x|z, y)).

Specifically, the generative process of the CVAE takes the form

$$( {\tilde{μ}}_{xy} , log {σ}_{xy}^{\sim 2} ) = g ( z , y ; ϕ ) , \qquad ( 2 )$$

$$q ( z \vert x , y ) = 𝒩 ( x ; {μ}_{xy} , diag ( {σ}_{xy}^{2} ) )$$

And the ELBO loss can then be written as:

$$\begin{matrix}{ℒ}_{ELBO} ( x \vert y ) = {E}_{q ( z , x , z )} [ log \frac{p ( z , x \vert y )}{q ( z \vert x , y )} ] \\ = {E}_{q ( z , x , y )} [ log p ( x \vert z , y ) + log p ( z \vert y ) - log q ( z \vert x , y ) ] ,\end{matrix} \qquad ( 3 )$$

In the CVAE model, the reconstruction of a sample is dependent on the given set of input conditions. To generate a TD-like output for an atypical sample, the conditional variable must be adjusted to a control condition while retaining the remaining conditions unchanged. Consequently, when calculating the discrepancy between the atypical input and the reconstructed output, the difference is assumed to be solely attributed to the modified conditions. This ensures that the identified divergence depends exclusively on the altered conditional variable.

### 2.5 Setup

Three commonly used VAE architectures in the fMRI domain were trained to learn a compact representation of the data from neurotypical control fMRI samples. A Convolutional Neural Network (CNN) variational autoencoder 113, 116, 115, Recurrent Neural Network (RNN) variational autoencoder 114, 117, 115, and a hybrid of CNN and RNN VAEs in parallel 113, 114, 116, 117, 115 (FIGS. 2, 11A). For all CNN VAEs in this disclosure, the CNN encoder 113 consisted of three convolution layers with 32, 64, and 128 filters, respectively, followed by a fully connected layer. Subsequently, the CNN decoder 116 comprised transposed convolution layers with 128, 64, and 32 filters, followed by a fully connected layer. Batch normalization and the leaky ReLU activation functions were utilized. The RNN encoder 114 contained three unidirectional Long-Short-Term Memory (LSTM) layers followed by a fully connected layer. RNN Decoder 117, respectively, includes a fully connected layer followed by three LSTM layers as well. Finally, the parallel structure model was built as a combination of those CNN and RNN structures in parallel. Latent features are fused using element-by-element multiplication.

A detailed summary of the structures of VAEs is shown in FIGS. 2A-2C, 11A. FIG. 2B corresponds to the CNN encoder layers 113 and CNN decoder layers 116 of FIG. 11A. And FIG. 2C corresponds to the RNN encoder layers 114 and RNN decoder layers 117 of FIG. 11A.

All three VAEs have 2000 latent features extracted by the encoding part (d=2000), and the latent space was modeled using a mixture of Gaussian assumptions. Furthermore, all VAEs were optimized using the Adam algorithm with a learning rate of 0.0001. In the context of the CVAE, all the architectures of the models remain the same; however, the phenotypic data embedding is incorporated by concatenating it with both the input of the encoder and the input of the decoder. The embedding dimensionality is specifically set to 200, allowing for concatenation as another parcel feature to the input matrix, resulting in a total dimensionality of 201×200. Concatenation to the latent vector z resulted in the dimensionality of 2200. It is important to note that for the training of VAEs, only a neurotypical sample has been used; however, due to the conditional embedding, the CVAE allows for training on both neurotypical and neurodivergent samples. All of the systems that are reported in this disclosure were performed on the server that contains an NVIDIA RTX 3090 running CUDA version 10.2 and PyTorch 1.13.1+cu117 [43]. This is the first system in the fMRI domain comparing different encoding and decoding architectures.

### 2.6 VAE Performance Evaluations

Evaluation of VAE performance consisted of analysis of the reconstruction of the neurotypical samples, analysis of latent space features, and analysis of the regeneration abilities of the decoder.

Upon completion of the training, assessment of the VAE and CVAE reconstruction abilities involved three evaluation methods. The cosine similarity score was computed to capture the overall resemblance between the input and the reconstructed output. However, cosine similarity does not explicitly account for positional information. Thus, Pearson's correlation coefficient (R, PCC) was additionally calculated for the validation subsets of the data. Finally, the difference between the input and decoded output was evaluated through L1 (Mean Absolute Error (MAE)). L1 quantified the average absolute difference between the reconstructed BOLD signal intensity and the intensity of the original signal. To compute the L1 error, we leveraged the validation samples of the subgroup present during the training phase. We believe that a combination of these metrics will help us quantify the ability of VAEs and CVAEs to reconstruct samples from lower-dimensional data within the validation dataset.

To assess the encoding abilities of each model, we encode both populations and conduct a comparative analysis of their latent representations. To determine the statistical significance of the differences in the encoding feature, a two-sided t-test is employed (p<0.05). The null hypothesis is that the mean of the neurotypical subgroup is equal to the mean of the neurodivergent. It is believed that the optimal encoder architecture will have a pronounced distinction in the latent space, meaning that the encoder learned to extract meaningful features from the input samples. Consequently, our objective is to reject the null hypothesis in favor of the alternative hypothesis, which is that the mean latent representations of the TD and ASD groups are different.

Evaluating the performance of accuracy of synthetic data outputted by VAEs poses a significant challenge, especially when the ground-truth effects are unknown in real data. Therefore, to provide an initial assessment of atypical pattern detection, we calculate L1 of synthetic samples. In the context of VAE systems, where the model is trained on TD samples only, we formulate a hypothesis that the L1 error would be more pronounced when reconstructing ASD validation samples in comparison to the TD validation samples. For the CVAE systems, where model architecture accommodates training on both TD and ASD samples, synthetic outputs were generated for the ASD validation dataset with target conditional embedding of TD samples. Consequently, the L1 error is computed between the input ASD samples and the synthetically generated outputs.

### 2.7 Functional Connectivity Analysis

In this system, we conducted FC analysis of the ASD subgroup alongside FC analysis for female and male populations within the ASD group. The FC analysis was performed using trained VAEs and CVAEs in three steps.

In VAE systems, we first processed each neurodivergent sample from the validation subset through all three architectures. We hypothesized that since VAEs were trained to reconstruct neurotypical samples only, the output of the neurodivergent sample from the decoding process would resemble the features of the training data (FIG. 4A). Next, we grouped the brain parcels into five prominent brain networks: the Default Mode Network (DMN), Limbic, Visual, Somatomotor, and Salience. Due to limitations in Schaefer's atlas, we could only analyze connectivity within these five networks. We then calculated pairwise connectivity using Pearson correlation coefficients between these networks (FIG. 4B). The resulting averaged correlation matrices were then subjected to a two-sided Welch t-test to compare interconnectivity within networks between the two subgroups. Statistically significant results (p<0.05) were then visualized using chord diagrams (FIG. 4C). A negative Welch t-value indicated that the mean of the neurodivergent input was lower than that of the neurotypical-like synthetically generated group, while a positive Welch t-value suggested that the mean of the input group was higher than the generated group. As depicted in FIG. 4C, the color of the connecting line between the outer circles of the chord diagram corresponds to the Welch t-value. In this representation, blue shades indicate negative t-values (lower connectivity), while yellow hues correspond to positive t-values (higher connectivity).

For the CVAEs, the training data included both neurodivergent and neurotypical data, which allows for a more targeted generation of the synthetic output. The overall steps for FC with CVAEs were similar to those with VAEs, but the input embedding of the condition was adjusted to the desired output. For instance, if the input sample was a female with ASD, 12 years old, the embedding was adjusted to generate a neurotypical-like female, 12 years old, sample. The remaining FC analysis steps (grouping parcels 204, calculating pairwise connectivity 214, conducting two-sided Welch t-tests 216, and visualizing chord diagrams 218 are the same as with VAEs.

To explore sex-related neurodivergence, we performed separate analyses for female and male samples from the validation dataset. To assess the influence of the conditions on the FC results, we calculate cosine similarity between VAE and CVAE pairwise correlation matrix between networks (FIG. 11B). We believe that the cosine similarity score should be higher for CVAE than VAE, indicating reduced sex-related bias.

### 3. RESULTS

### 3.1 VAE Performance Evaluations

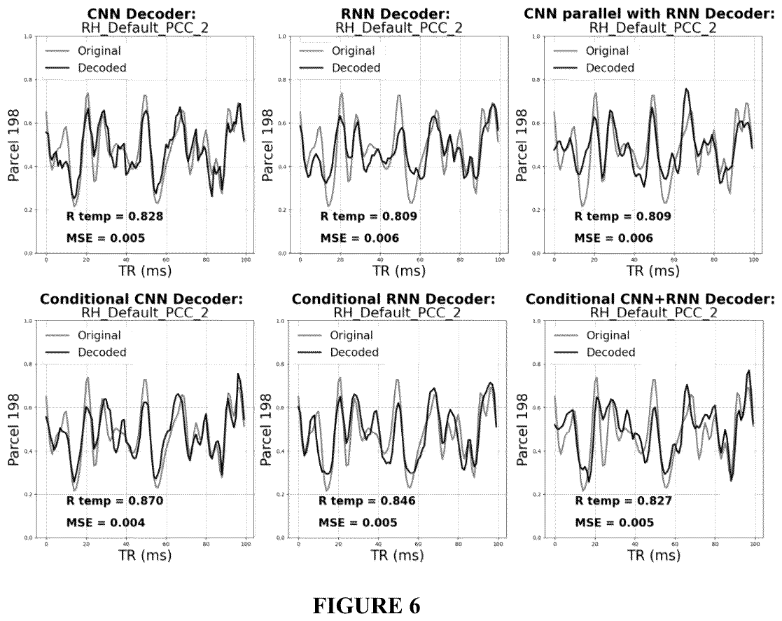

As detailed in Section 2.6, we begin by evaluating the reconstruction performance of all VAEs and CVAEs. Upon visual inspection of FIG. 5, we observe that all models have adeptly learned to reconstruct the data from the low-dimensional representation. In FIG. 6, one can observe the decoded signal from one parcel of the validation sample, and the decoded signal closely follows the input signal, demonstrating a high level of reconstruction. Additional quantitative results are summarized in Tables 1 and 2. It is worth highlighting that integrating conditional variables into the models has increased the accuracy in reconstructing latent features, as indicated by both the cosine similarity and PCC metrics. The CNN architecture has outperformed other architectures in terms of reconstruction across both the VAE and CVAE systems as evidenced by the highest PCC scores in Tables 1 and 2. Moreover, the increase in PCC by adding conditional embedding to the CNN model (9.37%) is higher compared to the increases in other models (4.54% in RNN and 3.18% in CNN+RNN).

TABLE 1

| | Cosine | | | |
|---|---|---|---|---|
| Model | Similarity | PCC | L1 TD | L1 ASD |
| CNN | 0.9930 | 0.6551 | 0.0693 | 0.0781 |
| RNN | 0.9817 | 0.6105 | 0.0728 | 0.0819 |
| CNN and RNN | 0.9820 | 0.6356 | 0.0717 | 0.0803 |

Table 1 is a summary of reconstruction performance of VAE systems: cosine similarity scores and PCC for the neurotypical samples in the validation dataset. The average L1 reconstruction error for both neurotypical and neurodivergent samples within the validation dataset is presented.

TABLE 2

| | Cosine | | | |
|---|---|---|---|---|
| Model | Similarity | PCC | L1 ASD | L1 TD_{synthetic} |
| Conditional | 0.9961 | 0.7165 | 0.0643 | 0.0733 |
| CNN | | | | |
| Conditional | 0.9818 | 0.6382 | 0.0681 | 0.077 |
| RNN | | | | |
| Conditional | 0.9825 | 0.6558 | 0.0687 | 0.0778 |
| CNN and RNN | | | | |

Table 2 is a summary of reconstruction performance of CVAE systems: cosine similarity scores and PCC for the neurotypical samples in the validation dataset. Additionally, the average L1 reconstruction error for validation neurodivergent samples and synthetically generated neurotypical-like samples.

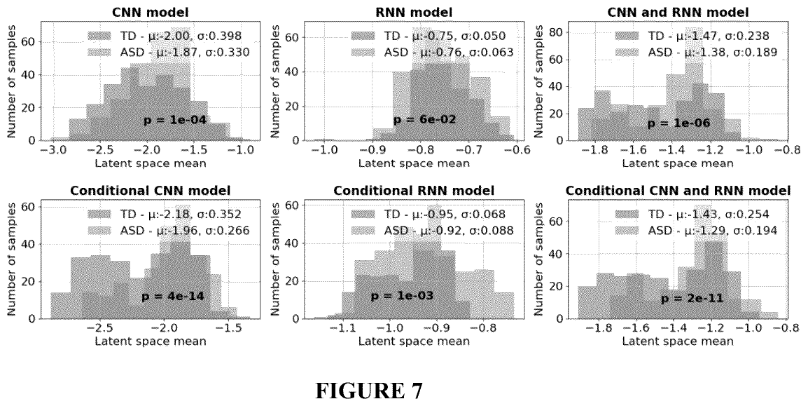

To evaluate the encoding capabilities of each model, a comprehensive analysis was conducted on both neurotypical and neurodivergent samples from the validation dataset. FIG. 7 depicts the resulting means of latent distribution. Notably, among the VAE models, the CNN architecture and the hybrid CNN with RNN models exhibit statistically significant differences in their latent features between affected and unaffected samples. Therefore, the models have successfully learned to extract meaningful features from the input data. As anticipated, adding conditional embedding to the models resulted in a higher degree of separation within the latent space than unconditional models. All the CVAE models display statistically significant differences in latent space between the two subgroups.

To further assess the performance of VAEs, we conducted a preliminary evaluation of atypical pattern detection by calculating the reconstruction error on both neurotypical and neurodivergent samples from our validation datasets, summarized in Table 1. The reconstruction L1 error for the ASD validation set is higher than that of the TD set. This difference implies that VAEs can reconstruct ASD samples in a manner that makes them resemble TD samples. For the CVAEs, we conducted a similar analysis. Given that the CVAE was trained on both ASD and TD samples, our approach involved computing the reconstruction L1 error for the ASD samples first. Subsequently, we compared this with the synthetically generated outputs, employing a target conditional embedding based on a TD sample. The results, presented in Table 2, show that the construction error for the synthetic samples exceeds that of the reconstructed ASD samples. This disparity serves as an indication that the conditioning mechanism is effective in detecting certain divergences within the data.

### 3.2. Functional Connectivity Analysis

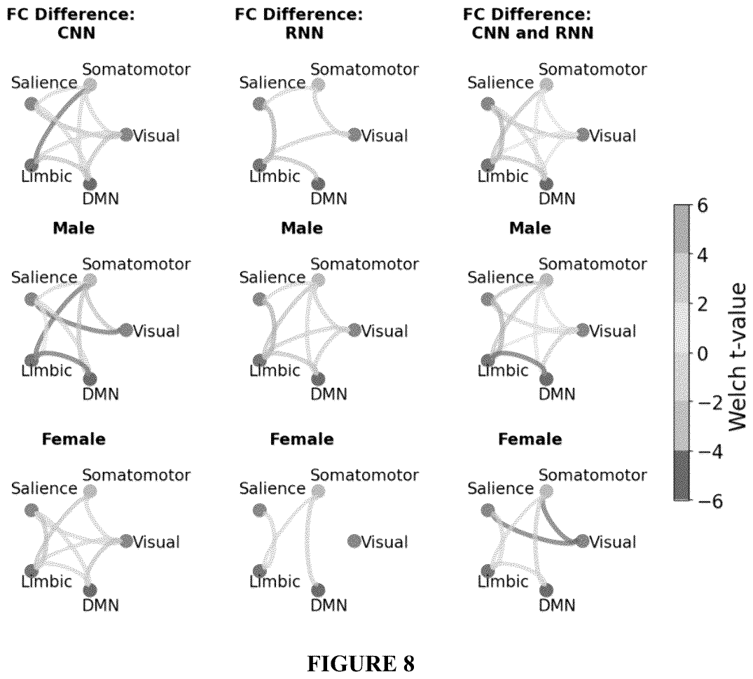

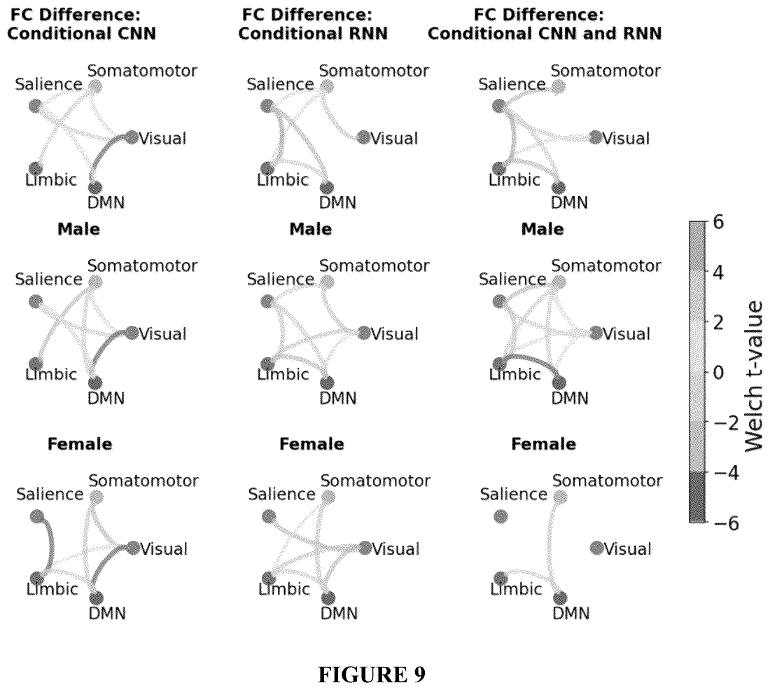

FIGS. 8 and 9 present the results of the FC analysis, following the steps outlined in Section 2.7. In FIGS. 8 and 9, the top row is the connectivity trends for both female and male samples of the testing data, the middle row is the trends for the male population, and the bottom is female. We first highlight results shared consistently across all rows, which indicates the trends that are unaffected by the sex imbalance within the dataset. Subsequently, we summarize the identified patterns in functional connectivity that were affected by this sex bias.

In the VAE systems (FIG. 8, top row), a consistent trend of under-connectivity between the Limbic and DMN networks emerges across all models for the combined male and female populations. This pattern remains evident in both the female and male subpopulations, except for the RNN female results (FIG. 8, bottom row). Similarly, CNN and hybrid models identified under-connectivity between the salience and visual networks, which has remained similarly apparent in both male and female populations. Finally, the trend that is found to be common across males subpopulation and females subpopulation (middle and bottom rows) is the under-connectivity between limbic and somatomotor networks.

One notable consequence of the dataset's bias is exemplified by the consistent trend of over-connectivity between the salience and limbic networks in the male population, which is reversed in females for all of the models (FIG. 8, middle and bottom rows). Thus, connectivity between the salience and limbic networks for the combined populations (top row) reveals contrasting outcomes. Furthermore, a noteworthy difference between males and females lies in the connectivity between the somatomotor and DMN networks. In females, the connectivity between the somatomotor and DMN networks exhibits a notably stronger presence in comparison to males. This can be concluded by higher Welch t-values observed within the female population for the connections between the somatomotor and DMN networks.

In the CVAE systems (FIG. 11A), some trends are similar to those identified with VAE models. For example, in FIG. 9, a trend of under-connectivity between limbic and DMN is apparent for both the male and female populations, with the exception of the CNN model. In RNN-based and hybrid models, the trend of under-connectivity between limbic and DMN in males and females remains true for CVAE systems. Remarkably, the trend of increased connectivity between the Somatomotor and DMN networks in females, as opposed to males, persists in CVAE systems as well.

Interpreting the chord plots and discerning the extent to which the CVAE mitigated sex-related influences presents a challenge. As outlined in Section 3.4, the identified neurodivergence in the CVAE is expected to have a lower correlation with sex labels compared to the VAE. To measure this, we quantitatively assess the similarity between the pairwise correlations underpinning these chord plots (Table 3). This similarity score revealed that all the conditional models have a higher overlap between male and female neurodivergence compared to the unconditional models. Conditional hybrid model had the highest values for the similarity between female and male pairwise correlation matrices, suggesting the most unbiased FC neurodivergence patterns in relation to sex. However, it is important to note that the CNN model compared to the rest of the models had the highest increase in similarity by adding conditional embedding, which is indicative of the fact that CNN layers are particularly sensitive to the inclusion of conditional embedding.

Table 3 shows the similarity between male and female FC pairwise correlations for VAE and CVAE systems.

TABLE 3

| | Unconditional | Conditional |
|---|---|---|
| Model Architecture | FC Similarity | FC Similarity |
| CNN | 0.35 | 0.70 |
| RNN | 0.66 | 0.80 |
| CNN parallel with RNN | 0.78 | 0.85 |

### 4. DISCUSSION

In this system 100, we investigated the application of generative models to FC analysis in the context of ASD with fMRI data. Our exploration began with a comprehensive assessment of the reconstructive abilities of various VAE architectures, using neurotypical samples as the input data.

The CNN-based VAE and CVAE are more effective in reconstruction and conditional generation of synthetic data. This conclusion has been derived from the combination of the metrics and results provided in Section 3. First and foremost, cosine similarity and PCC measures for CNN VAE and CNN CVAE reconstruction are higher compared to the other models. Even though introducing phenotype data has improved both reconstruction in higher dimensional space and discrimination in lower-dimensional space for all of the models, it is worth highlighting that the CNN model has demonstrated superior reconstruction performance with conditioning, as evidenced by the highest observed increase in PCC by comparing Tables 1 and 2.

Secondly, in Table 3, the CNN model stands out with the highest increase in similarity between female and male connectivity, almost doubling the improvement seen in other models. These findings collectively indicate that the CNN model exhibits heightened sensitivity to conditioning mechanisms in comparison to other models.

One plausible explanation for this could lie in the way the condition is introduced to the model. In all models, conditional embedding is concatenated as another feature, resulting in the absence of explicit temporal ordering within conditional embeddings. Consequently, CNN demonstrated superior performance in handling this conditioning mechanism when contrasted with the RNN model. We interpret this as it is more effective for the VAE to model spatial patterns rather than temporal ones. We believe that unconditional CNN in parallel with RNN is better for classification applications.

As shown in FIG. 7, the degree of separation for the unconditional hybrid model is higher than the other models. Secondly, the addition of conditional information to the hybrid model resulted in the most unbiased results in relation to sex labels results, as indicated by the highest values in Table 3. Nevertheless, the hybrid model has reduced sex-related bias the most, the results were accompanied by poorer reconstruction performance compared to the CNN-based model.

To provide initial validation for the decoder architectures, we calculated the Mean Absolute Error for the reconstruction of the subgroup that was present during the training and the new sample subgroup. VAEs had higher reconstruction errors for ASD samples compared to TD samples, indicating their ability to model ASD samples resembling TD ones. For CVAEs, which were trained on both ASD and TD samples, we computed reconstruction loss for ASD samples. Comparing this loss of synthetically generated outputs using a TD-based target conditional embedding, we found higher reconstruction errors for synthetic samples. This finding also suggests the conditioning mechanism effectively detects neurodivergence and can make the generation process more targeted. Moreover, in comparison to the work of Kim et al. [22], our VAE models demonstrate better performance in data reconstruction, showcasing a lower range of reconstruction errors ranging from 0.06 to 0.07, while Kim et al. reported a range of 1.2 to 1.7.

Next, we proceeded further to FC analysis with trained VAEs and CVAEs. We consistently identified under-connectivity between the limbic and DMN networks across most VAE systems, which is consistent with previous findings in the literature. The trend of higher connectivity between salience and limbic networks in the male population compared to female has been identified by all VAEs and CVAEs. In the study by Green et al. [42], where the studied group consisted predominantly of the male population, they concluded that male individuals tend to exhibit over-connectivity between salience and limbic networks. However, we extend these findings and show this trend does not hold true in the female population.

One of the findings in the previous literature is that males tend to have decreased under-connectivity with the DMN network compared to females [44]. Based on our analysis, both the VAE and CVAE revealed this pattern as well, specifically between DMN and somatomotor networks. Due to the limitations of Schaefer's atlas, we have focused on exploring network connectivity. However, in future works, we aim to expand the investigation to other atlas configurations and within network analysis. All of the above are consistent with the connectivity patterns that have been reported previously in other literature, summarized in Section 2.4.

It was hypothesized that adjusting conditional embedding would reduce sex-related bias in the models and potentially result in sex-independent FC. By evaluating the pairwise connectivity matrix overlap between female and male subgroups, it is concluded that patterns discerned through CVAE have reduced correlation with sex labels. We believe the remaining differences shown in the chord plot between male and females in CVAE systems are primarily due to the age difference and diverse nature of the disorder.

In recent years, many studies have explored the capabilities of generative models (including VAEs, GANs, and Diffusion flow models) in the medical domain. However, many models are found to struggle with at least one of the following: high-quality outputs, mode coverage, sample diversity, and computational costs [50]. VAEs are probabilistic models, which makes them well-suited for modeling and generating complex distributions. As shown in this disclosure, VAEs can learn the underlying probability distribution of the input data, allowing for probabilistic sampling and interpolation. However, as stated in previous works, VAEs tend to suffer from comparatively low quality in generation compared to GANs or Diffusion flow models [50]. Therefore, our future work will also investigate different generative frameworks to improve the quality of generated samples and develop methods for assessing them.

### 5. CONCLUSIONS

The present system and method present a novel approach to FC analysis of fMRI data using a generative model such as the VAE. We also determined if introducing additional phenotype data to the model would reduce bias and increase the generalizability of the FC analysis. Our main finding includes that the CNN-based model 113, 116, 115 has been shown to be the most effective architecture for the FC analysis, as it showed superior performance in reconstruction with and without conditional information. We show that introducing phenotypic data as conditional embeddings to the model generally improves reconstruction performance and reduces bias in FC analysis. Conditioning of the CNN model has shown to have the most effect on the results; however, the CNN model parallel with RNN (FIG. 11A) has shown to be the least biased with respect to sex labels.

The following abbreviations are used in this manuscript:
ASD Autism Spectrum Disorder
TD Typically Developing
FC Functional Connectivity
fMRI functional Magnetic Resonance Imaging
BOLD Blood-Oxygen-Level-Dependent
CNN Convolutional Neural Network RNN Recurrent Neural Network
VAE Variational AutoEncoder
CVAE Conditional Variational AutoEncoder
DMN Default Mode Network
PCC Pearson's Correlation Coefficient

The following references are hereby incorporated by reference.
1. Cherkassky, V. L.; Kana, R. K.; Keller, T. A.; Just, M. A. Functional connectivity in a baseline resting-state network in autism. Neuroreport 2006, 17, 1687-1690. https://doi.org/10.1097/01.wnr.0000239956.45448.4c.
2. Calhoun, V. D.; Liu, J.; Adalκ, T. A review of group ICA for fMRI data and ICA for joint inference of imaging, genetic, and ERP data. Neuroimage 2009, 45, S163-S172. https://doi.org/10.1016/j.neuroimage.2008.10.057.
3. Farahani, F. V.; Karwowski, W.; Lighthall, N. R. Application of graph theory for identifying connectivity patterns in human brain networks: A systematic review. Front. Neurosci. 2019, 13, 585. https://doi.org/10.3389/fnins.2019.00585.
4. Chen, G.; Ward, B. D.; Xie, C.; Li, W.; Chen, G.; Goveas, J. S.; Antuono, P. G.; Li, S. J. A clustering-based method to detect functional connectivity differences. NeuroImage 2012, 61, 56-61. https://doi.org/10.1016/j.neuroimage.2012.02.064.
5. Hutchison, R. M.; Womelsdorf, T.; Allen, E. A.; Bandettini, P. A.; Calhoun, V. D.; Corbetta, M.; Della Penna, S.; Duyn, J. H.; Glover, G. H.; Gonzalez-Castillo, J.; et al. Dynamic functional connectivity: Promise, issues, and interpretations. Neuroimage 2013, 80, 360-378. https://doi.org/10.1016/j.neuroimage.2013.05.079.
6. Havlicek, M.; Jan, J.; Brazdil, M.; Calhoun, V. D. Dynamic Granger causality based on Kalman filter for evaluation of functional network connectivity in fMRI data. Neuroimage 2010, 53, 65-77. https://doi.org/10.1016/j.neuroimage.2010.05.063.
7. Stephan, K. E.; Kasper, L.; Harrison, L. M.; Daunizeau, J.; den Ouden, H. E.; Breakspear, M.; Friston, K. J. Nonlinear Dynamic Causal Models for fMRI. Neuroimage 2008, 42, 649-662. https://doi.org/10.1016/j.neuroimage.2008.04.262.
8. Kingma, D. P.; Welling, M. Auto-Encoding Variational Bayes. arXiv 2022, arXiv.1312.6114. https://doi.org/10.48550/arXiv.1312.6 114.
9. Sohn, K.; Lee, H.; Yan, X. Learning Structured Output Representation using Deep Conditional Generative Models. In Advances in Neural Information Processing Systems; Cortes, C., Lawrence, N., Lee, D., Sugiyama, M., Garnett, R., Eds.; Curran Associates, Inc.: Red Hook, NY, USA, 2015; Volume 28.
10. Lee, M. H.; Smyser, C. D.; Shimony, J. S. Resting-state fMRI: A review of methods and clinical applications. Am. J. Neuroradiol. 2013, 34, 1866-1872. https://doi.org/10.3174/ajnr.A3263.
11. Neha.; Gandhi, T. K. Resting state fMRI analysis using seed based and ICA methods. In Proceedings of the 2016 3rd International Conference on Computing for Sustainable Global Development (INDIACom), New Delhi, India, 16-18 Mar. 2016; pp. 2551-2554.
12. Erhardt, E. B.; Rachakonda, S.; Bedrick, E. J.; Allen, E. A.; Adali, T.; Calhoun, V. D. Comparison of multi-subject ICA methods for analysis of fMRI data. Hum. Brain Mapp. 2011, 32, 2075-2095. https://doi.org/10.1002/hbm.21170.
13. Almuqhim, F.; Saeed, F. ASD-SAENet: A sparse autoencoder, and deep-neural network model for detecting autism spectrum disorder (ASD) using fMRI data. Front. Comput. Neurosci. 2021, 15, 654315. https://doi.org/10.3389/fncom.2021.654315.
14. Kang, L.; Chen, J.; Huang, J.; Jiang, J. Autism spectrum disorder recognition based on multi-view ensemble learning with multi-site fMRI. Cogn. Neurodynamics 2023, 17, 345-355. https://doi.org/10.1007/s11571-022-09828-9.
15. Qiang, N.; Dong, Q.; Liang, H.; Ge, B.; Zhang, S.; Sun, Y.; Zhang, C.; Zhang, W.; Gao, J.; Liu, T. Modeling and augmenting of fMRI data using deep recurrent variational auto-encoder. J. Neural Eng. 2021, 18, 0460b6. https://doi.org/10.1088/1741-2552/ac1179.
16. Kim, J. H.; Zhang, Y.; Han, K.; Wen, Z.; Choi, M.; Liu, Z. Representation learning of resting state fMRI with variational autoencoder. NeuroImage 2021, 241, 118423. https://doi.org/10.1016/j.neuroimage.2021.118423.
17. Huang, H.; Hu, X.; Zhao, Y.; Makkie, M.; Dong, Q.; Zhao, S.; Guo, L.; Liu, T. Modeling task fMRI data via deep convolutional autoencoder. IEEE Trans. Med. Imaging 2017, 37, 1551-1561. https://doi.org/10.1109/TMI.2017.2715285.
18. Zuo, Q.; Zhu, Y.; Lu, L.; Yang, Z.; Li, Y.; Zhang, N. Fusing Structural and Functional Connectivities using Disentangled VAE for Detecting MCI. arXiv 2023, arXiv: 2306.09629.
19. Choi, H. Functional connectivity patterns of autism spectrum disorder identified by deep feature learning. arXiv 2017, arXiv: 1707.07932. https://doi.org/10.48550/arXiv.1707.07932.
20 Zhuang, P.; Schwing, A. G.; Koyejo, O. fMRI Data Augmentation Via Synthesis. In Proceedings of the 2019 IEEE 16th International Symposium on Biomedical Imaging (ISBI 2019), Venice, Italy, 8-11 Apr. 2019; IEEE: Piscataway, NJ, USA, 2019; pp. 1783-1787. https://doi.org/10.1109/ISBI.2019.8759585.
21. Zhang, Y.; Liu, X.; Wa, S.; Liu, Y.; Kang, J.; Lv, C. GenU-Net++: An Automatic Intracranial Brain Tumors Segmentation Algorithm on 3D Image Series with High Performance. Symmetry 2021, 13, 2395. https://doi.org/10.3390/sym13122395.
22. Tashiro, T.; Matsubara, T.; Uehara, K. Deep neural generative model for fMRI image based diagnosis of mental disorder. IEEE Proc. Ser. 2017, 29, 700-703. https://doi.org/10.34385/proc.29.C2L-D-6.
23. Zou, A.; Ji, J. Learning brain effective connectivity networks via controllable variational autoencoder. In Proceedings of the 2021 IEEE International Conference on Bioinformatics and Biomedicine (BIBM), Houston, TX, USA, 9-12 Dec. 2021; pp. 284-287. https://doi.org/10.1109/BIBM52615.2021.9669871.
24. Wang, X.; Zhao, K.; Zhou, R.; Leow, A.; Osorio, R.; Zhang, Y.; He, L. Normative Modeling via Conditional Variational Autoencoder and Adversarial Learning to Identify Brain Dysfunction in Alzheimer's Disease. arXiv 2022, arXiv.2211.08982. https://doi.org/10.48550/arXiv.2211.08982.
25. Gao, M. S.; Tsai, F. S.; Lee, C. C. Learning a Phenotypic-Attribute Attentional Brain Connectivity Embedding for ADHD Classification using rs-fMRI. In Proceedings of the 2020 42nd Annual International Conference of the IEEE Engineering in Medicine & Biology Society (EMBC), Montreal, QC, Canada 20-24 Jul. 2020; pp. 5472-5475. https://doi.org/10.1109/EMBC44109.2020.9175789.
26. Broyd, S. J.; Demanuele, C.; Debener, S.; Helps, S. K.; James, C. J.; Sonuga-Barke, E. J. Default-mode brain dysfunction in mental disorders: A systematic review. Neurosci. Biobehav. Rev. 2009, 33, 279-296. https://doi.org/10.1016/j.neubiorev.2008.09.002.
27. Rajmohan, V.; Mohandas, E. The Limbic System. Indian J. Psychiatry 2007, 49, 132.
28 Rolls, E. T. The cingulate cortex and limbic systems for emotion, action, and memory. Brain Struct. Funct. 2019, 224, 3001-3018. https://doi.org/10.1007/s00429-019-01945-2.
29. Yang, Y. L.; Deng, H. X.; Xing, G. Y.; Xia, X. L.; Li, H. F. Brain functional network connectivity based on a visual task: Visual information processing-related brain regions are significantly activated in the task state. Neural Regen. Res. 2015, 10, 298-307. https://doi.org/10.4103/1673-5374.152386.
30. Menon, V.; Uddin, L. Q. Saliency, switching, attention and control: A network model of insula function. Brain Struct. Funct. 2010, 214, 655-667. https://doi.org/10.1007/s00429-010-0262-0.
31. Chand, G. B.; Dhamala, M. The salience network dynamics in perceptual decision-making. Neuroimage 2016, 134, 85-93. https://doi.org/10.1016/j.neuroimage.2016.04.018.
32. Abrams, D. A.; Lynch, C. J.; Cheng, K. M.; Phillips, J.; Supekar, K.; Ryali, S.; Uddin, L. Q.; Menon, V. Underconnectivity between voice-selective cortex and reward circuitry in children with autism. Proc. Natl. Acad. Sci. USA 2013, 110, 12060-12065. https://doi.org/10.1073/pnas.1302982110.
33. Lynch, C. J.; Uddin, L. Q.; Supekar, K.; Khouzam, A.; Phillips, J.; Menon, V. Default mode network in childhood autism: Posteromedial cortex heterogeneity and relationship with social deficits. Biol. Psychiatry 2013, 74, 212-219. https://doi.org/10.1 016/j.biopsych.2012.12.013.
34 Yerys, B. E.; Gordon, E. M.; Abrams, D. N.; Satterthwaite, T. D.; Weinblatt, R.; Jankowski, K. F.; Strang, J.; Kenworthy, L.; Gaillard, W. D.; Vaidya, C. J. Default mode network segregation and social deficits in autism spectrum disorder: Evidence from non-medicated children. NeuroImage Clin. 2015, 9, 223-232. https://doi.org/10.1016/j.nicl.2015.07.018.
35. Buch, A. M.; Vértes, P. E.; Seidlitz, J.; Kim, S. H.; Grosenick, L.; Liston, C. Molecular and network-level mechanisms explaining individual differences in autism spectrum disorder. Nat. Neurosci. 2023, 26, 650-663. https://doi.org/10.1038/s41593-023-01259-x.
36. Green, S. A.; Hernandez, L.; Bookheimer, S. Y.; Dapretto, M. Salience network connectivity in autism is related to brain and behavioral markers of sensory overresponsivity. J. Am. Acad. Child Adolesc. Psychiatry 2016, 55, 618-626. https://doi.org/10.101 6/j.jaac.2016.04.013.
37. Ypma, R. J.; Moseley, R. L.; Holt, R. J.; Rughooputh, N.; Floris, D. L.; Chura, L. R.; Spencer, M. D.; Baron-Cohen, S.; Suckling, J.; Bullmore, E. T.; et al. Default Mode Hypoconnectivity Underlies a Sex-Related Autism Spectrum. Biol. Psychiatry: Cogn. Neurosci. Neuroimaging 2016, 1, 364-371. https://doi.org/10.1016/j.bpsc.2016.04.006.
38 Lawrence, K. E.; Hernandez, L. M.; Bowman, H. C.; Padgaonkar, N. T.; Fuster, E.; Jack, A.; Aylward, E.; Gaab, N.; Van Horn, J. D.; Bernier, R. A.; et al. Sex differences in functional connectivity of the salience, default mode, and central executive networks in youth with ASD. Cereb. Cortex 2020, 30, 5107-5120. https://doi.org/10.1093/cercor/bhaa105.
39 Smith, R. E.; Avery, J. A.; Wallace, G. L.; Kenworthy, L.; Gotts, S. J.; Martin, A. Sex differences in resting-state functional connectivity of the cerebellum in autism spectrum disorder. Front. Hum. Neurosci. 2019, 13, 104. https://doi.org/10.3389/fnhum.2019.00104.
40. Craddock, C.; Benhajali, Y.; Chu, C.; Chouinard, F.; Evans, A.; Jakab, A.; Khundrakpam, B. S.; Lewis, J. D.; Li, Q.; Milham, M.; et al. The neuro bureau preprocessing initiative: Open sharing of preprocessed neuroimaging data and derivatives. Front. Neuroinform. 2013, 7, 27.
41 Schaefer, A.; Kong, R.; Gordon, E. M.; Laumann, T. O.; Zuo, X. N.; Holmes, A. J.; Eickhoff, S. B.; Yeo, B. T. Local-global parcellation of the human cerebral cortex from intrinsic functional connectivity MRI. Cereb. Cortex 2018, 28, 3095-3114.
42. Rumelhart, D. E.; Hinton, G. E.; Williams, R. J. Learning Internal Representations by Error Propagation. 1985.
43. Paszke, A.; Gross, S.; Massa, F.; Lerer, A.; Bradbury, J.; Chanan, G.; Killeen, T.; Lin, Z.; Gimelshein, N.; Antiga, L.; et al. PyTorch: An Imperative Style, High-Performance Deep Learning Library. In Advances in Neural Information Processing Systems 32; Curran Associates, Inc.: Red Hook, NY, USA, 2019; pp. 8024-8035.
44. Xiao, Z.; Kreis, K.; Vahdat, A. Tackling the generative learning trilemma with denoising diffusion gans. arXiv 2021, arXiv: 2112.07804. https://doi.org/10.48550/arXiv.2112.07804.

In the embodiments shown, a processing device can be provided to perform various functions and operations in accordance with the disclosure. The processing device can be, for instance, a computer, personal computer (PC), server or mainframe computer, or more generally a computing device, processor, application specific integrated circuits (ASIC), or controller. The processing device can be provided with, or be in communication with, one or more of a wide variety of components or subsystems including, for example, data processing devices and subsystems, wired or wireless communication links, user-actuated (e.g., voice or touch actuated) input devices (such as touch screen, keyboard, mouse) for user control or input, monitors for displaying information to the user, and/or storage device(s) such as memory, RAM, ROM, DVD, CD-ROM, analog or digital memory, flash drive, database, computer-readable media, floppy drives/disks, and/or hard drive/disks. All or parts of the system, processes, and/or data utilized in the system of the disclosure can be stored on or read from the storage device(s). The storage device(s) can have stored thereon machine executable instructions for performing the processes of the disclosure. The processing device can execute software that can be stored on the storage device. Unless indicated otherwise, the process is preferably implemented automatically by the processor substantially in real time without delay.

As used herein, when an element or feature is described as being “configured,” that element or feature is structurally arranged or formed to accomplish the stated purpose. As used with respect to a processing device (e.g., computer), the term “configured” or “configured to” means that the processing device is structurally arranged or ordered (e.g., by supplying, arranging or connecting a specific set of internal or external components or modules, for example that perform certain operations) to accomplish the stated purpose or task.

The description and drawings of the present disclosure provided in the disclosure should be considered as illustrative only of the principles of the disclosure. The disclosure may be configured in a variety of ways and is not intended to be limited by the preferred embodiment. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A computer-implemented method for identifying neurodivergences in a patient using functional Magnetic Resonance Imaging (fMRI) data of a patient, the method comprising:
mapping at a processing device, the patient fMRI data of the patient to a predefined brain atlas;
applying at the processing device, a conditional variational autoencoder (CVAE) model to the mapped fMRI data, wherein the CVAE model is conditioned on sex, age, and neurodivergence subgroup label;
generating at the processing device, neurodivergent synthetic fMRI data corresponding to the conditions of sex, age and neurodivergence subgroup label using the CVAE model;
generating at the processing device, non-neurodivergent synthetic fMRI data corresponding to sex and age conditions using the CVAE model; and
conducting at the processing device, functional connectivity analysis to identify differences between the neurodivergent synthetic fMRI data and the non-neurodivergent synthetic fMRI data to identify neurodivergences in the patient fMRI data.

2. The method of claim 1, wherein the neurodivergence comprises Autism Spectrum Disorder (ASD).

3. The method of claim 1, wherein the functional connectivity analysis comprises:
calculating correlation matrices based on the neurodivergent synthetic fMRI data and the non-neurodivergent synthetic fMRI data; and
applying Welch t-test to the correlation matrices to identify the differences in functional connectivity patterns between patient fMRI data.

4. The method of claim 1, wherein the CVAE model optimizes latent space representations of the fMRI data to enhance the separability of individuals with ASD and healthy individuals.

5. The method of claim 1, wherein the CVAE model is trained using a deep learning architecture having multiple layers of neural networks.

6. The method of claim 1, further comprising displaying at a display device a chord diagram indicating connectivity between networks.

7. The method of claim 1, wherein the fMRI data is a 3-dimensional volumetric image of the patient's brain taken over time and represents brain activity of the patient's brain.

8. The method of claim 7, wherein the fMRI data is captured by a Magnetic Resonance Imaging (MRI) device.

9. The method of claim 1, wherein the functional connectivity analysis estimates different neural expressions with conditions and clinical progression.

10. The method of claim 1, wherein said mapping determines a brain region of the fMRI data to identify an area of interest and functional differences.

11. The method of claim 1, wherein the subgroup neurodivergence label including: normal, autism, and epilepsy.

12. The method of claim 1, wherein said CVAE model comprises a neural network architecture.

13. A non-transitory computer-readable storage medium comprising instructions that, when executed by a computer, cause the computer to perform the method of claim 1.

14. A computer system configured to identify neurodivergences in a patient, the system comprising:
a feature extraction module configured to receive patient fMRI data and extract features from the patient fMRI data;
a conditional variational autoencoder (CVAE) model conditioned on sex, age, and diagnosis, to generate synthetic patient fMRI data based on the patient fMRI data; and
a functional connectivity analysis module configured to perform functional connectivity analysis on the synthetic fMRI data to identify neurodivergences in individuals.

15. The system of claim 14, further comprising a data collection module configured to collect fMRI data from individuals.

16. A system for image classification and inventory management, the system comprising:
one or more non-transitory memory devices; and
one or more hardware processors configured to execute instructions from the one or more non-transitory memory devices to cause the system to:
map fMRI data to a predefined brain atlas;
obtaining a trained conditional variational autoencoder (CVAE) model based on extracted features, wherein the CVAE model is conditioned on sex, age, and subgroup label;
generate synthetic fMRI data using the trained CVAE model; and
perform functional connectivity analysis on the synthetic fMRI data to identify neurodivergences in the fMRI data.
